(19) United States
(12) Patent Application Publication
(10) Pub. No.: US 2023/0290017 A1
(43) Pub. Date: Sep. 14, 2023

(54) SKIN CHARACTERISTIC DATA VISUALIZATION DEVICE AND SYSTEM

(71) Applicant: LG HOUSEHOLD & HEALTH CARE LTD., Seoul (KR)

(72) Inventors: Hyung Kyu KIM, Seoul (KR); Jong Wook KIM, Seoul (KR); Oh Sun KWON, Seoul (KR)

(73) Assignee: LG HOUSEHOLD & HEALTH CARE LTD., Seoul (KR)

(21) Appl. No.: 18/120,622

(22) Filed: Mar. 13, 2023

(30) Foreign Application Priority Data
Mar. 14, 2022 (KR) 10-2022-0031365

(51) Int. Cl. G06T 11/00; H04N 23/63; G06T 7/73

(52) U.S. Cl. CPC G06T 11/001; H04N 23/632; G06T 7/73; G06T 2200/24; G06T 2207/30201

(57) ABSTRACT

A skin characteristic data visualization system according to an embodiment of the present disclosure may include a measuring device and a skin characteristic data visualization device communicating with the measuring device, wherein the skin characteristic data visualization device may include a camera, a display displaying a preview image of the camera, and a processor acquiring a measurement point which is a position of a skin to be measured by a measuring device, wherein the processor may display the measurement point on the preview image by an augmented reality (AR).

### CROSS-REFERENCE TO RELATED APPLICATIONS

This application claims priority to and the benefit of Korean Patent Application No. 10-2022-0031365, filed on Mar. 14, 2022, the disclosure of which is incorporated herein by reference in its entirety.

### TECHNICAL FIELD

The present disclosure relates to a device and system for visualizing skin characteristic data.

### BACKGROUND

In the past, expression of skin characteristics was limited to text or graphs due to technical difficulties. However, recently, people prefer image-oriented content rather than text and want intuitive understanding. Users are tired of text-oriented product advertisements and react sensitively to advertisements with visual effects.

Therefore, there is a need for a technique in which the user's skin characteristics, product effects, and the like are visually expressed.

### SUMMARY

### Technical Problem

The present disclosure is directed to providing a skin characteristic data visualization device and system that implements visualization data representing skin characteristics on a real facial image in real time through augmented reality (AR) technology.

The present disclosure is directed to providing a skin characteristic data visualization device and system that implements skin condition changes over time as an image through a timeline.

### Technical Solution

A skin characteristic data visualization device according to an embodiment of the present disclosure may include a camera, a display displaying a preview image of the camera, and a processor that acquires a measurement point which is a position of a skin to be measured by a measuring device, wherein the processor may display the measurement point on the preview image by an augmented reality (AR).

A skin characteristic data visualization system according to an embodiment of the present disclosure may include a measuring device and a skin characteristic data visualization device communicating with the measuring device, wherein the skin characteristic data visualization device may include a camera, a display displaying a preview image of the camera, and a processor acquiring a measurement point which is a position of a skin to be measured by a measuring device, wherein the processor may display the measurement point on the preview image by an augmented reality (AR).

The processor may change a color of the measurement point when the measuring device is positioned at the measurement point.

The processor may further display a position of the measuring device on the preview image by the AR.

The processor may activate a window for inputting a measurement value when the measuring device is positioned at the measurement point and deactivate a window for inputting the measurement value when the measuring device is not positioned at the measurement point.

The processor may further display past data when there is a past measurement record at the measurement point where the measuring device is positioned.

The processor may display a number of measurement points differently according to a user's face size on the preview image.

The processor may further display a simulation image representing a change in skin characteristics according to a past measurement date and a measurement value.

The processor may further display monitor information together with the simulation image, and the monitor information may include external environment information and customer information.

When displaying the simulation image, the processor may set a transition effect between scenes.

The skin characteristic data visualization device may further include a communication unit wirelessly connected to the measuring device, and the processor may receive measurement information corresponding to the measuring point from the measuring device.

### Advantageous Effects

According to an embodiment of the present disclosure, since skin characteristics are visualized on an actual user's facial image by an AR, there is an advantage that it is possible to intuitively understand the skin characteristics and product effects.

According to the embodiment of the present disclosure, since a measurement point is displayed on the actual user's facial image by the AR, there is an advantage that the ease and accuracy of skin measurement are improved.

### BRIEF DESCRIPTION OF THE DRAWINGS

FIG. 1 is a control block diagram of a skin characteristic data visualization system according to an embodiment of the present disclosure;

FIG. 2 is a block diagram illustrating a detailed configuration of a server of FIG. 1;

FIG. 3 is a control block diagram of a skin characteristic data visualization device according to an embodiment of the present disclosure;

FIG. 4 is a flowchart illustrating an operating method of the skin characteristic data visualization device according to the embodiment of the present disclosure;

FIG. 5 is a flowchart illustrating a method in which the skin characteristic data visualization device acquires a measurement point;

FIG. 6 is a flowchart illustrating a method of acquiring measurement information by the skin characteristic data visualization device according to the embodiment of the present disclosure;

FIG. 7 is a flowchart illustrating a method of displaying measurement information by the skin characteristic data visualization device according to the embodiment of the present disclosure;

FIG. 8 is an example of a screen displaying measurement points in an AR by the skin characteristic data visualization device according to the embodiment of the present disclosure;

FIG. 9 is an example of a screen in which the skin characteristic data visualization device according to the embodiment of the present disclosure displays a measurement point, a measurement region, and a position of a measurement device by the AR;

FIG. 10 is an exemplary view illustrating a screen on which the skin characteristic data visualization device according to the embodiment of the present disclosure displays past data;

FIG. 11 is a diagram illustrating a state in which the skin characteristic data visualization device according to the embodiment of the present disclosure varies the number of measurement points according to a face size;

FIG. 12 is an example of a screen on which measurement information is displayed in the skin characteristic data visualization device according to the embodiment of the present disclosure;

FIG. 13 is a diagram illustrating a state in which the skin characteristic data visualization device according to the embodiment of the present disclosure displays measurement information for each period in a timeline;

FIG. 14 is a diagram illustrating a state in which the skin characteristic data visualization device according to the embodiment of the present disclosure displays a simulation;

FIG. 15 is a diagram illustrating a state in which the skin characteristic data visualization device according to the embodiment of the present disclosure displays the simulation together with monitor information;

FIG. 16 is a diagram illustrating a state in which a skin characteristic data visualization device according to an embodiment of the present disclosure displays a monitor information icon;

FIG. 17 is a diagram illustrating a method of receiving task information by the skin characteristic data visualization device according to the embodiment of the present disclosure;

FIG. 18 is a diagram illustrating a method of receiving test subject information by the skin characteristic data visualization device according to the embodiment of the present disclosure;

FIG. 19 is a diagram illustrating a method of displaying a data skin list by the skin characteristic data visualization device according to the embodiment of the present disclosure;

FIG. 20 is an exemplary view of a data skin information input pop-up displayed by the skin characteristic data visualization device according to the embodiment of the present disclosure;

FIG. 21 is an exemplary diagram illustrating a method of setting a timeline by the skin characteristic data visualization device according to the embodiment of the present disclosure; and

FIG. 22 is an exemplary diagram illustrating a method of setting a simulation by the skin characteristic data visualization device according to the embodiment of the present disclosure.

### DETAILED DESCRIPTION OF THE EMBODIMENTS

Hereinafter, embodiments disclosed in the present specification will be described in detail with reference to the accompanying drawings, however, the same elements are designated by the same reference numerals, and repeated description thereof will be omitted.

Suffixes “module” and “part” for elements used in the following descriptions are given or used just for convenience in writing the specification, and do not have meanings or roles distinguishable between them.

In addition, in describing embodiments of the present disclosure, when detailed description of a known function is deemed to unnecessarily blur the gist of the present disclosure, the detailed description will be omitted. Further, accompanying drawings are only for easily understanding embodiments disclosed in the present disclosure, and the technical spirit disclosed in the present disclosure are not limited by the accompanying drawings, and it should be understood that the present invention includes all modifications, equivalents, and alternatives falling within the spirit and scope of the claims.

The terms including an ordinal number such as first, second, and the like may be used to describe various elements, but the elements are not limited by the terms. The terms are only used to distinguish one element from another.

The singular expression includes the plural expression unless the context clearly dictates otherwise.

It should be understood that the terms such as “comprises”, “have”, or the like, when used in the present application, specify the presence of stated features, integers, steps, operations, elements, and/or components, but do not preclude the presence or addition of one or more other features, integers, steps, operations, elements, components, and/or groups thereof.

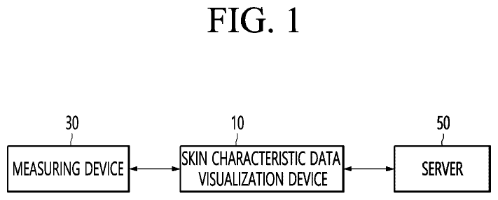

FIG. 1 is a control block diagram of a skin characteristic data visualization system according to an embodiment of the present disclosure.

As shown in FIG. 1, the skin characteristic data visualization system may include at least some or all of a skin characteristic data visualization device 10, a measuring device 30, and a server 50.

The skin characteristic data visualization device 10 may visually output skin characteristic data. The skin characteristic data visualization device 10 may be installed to be fixed at a specific position or implemented in a form of a movable device. Alternatively, the skin characteristic data visualization device 10 may be implemented in a form of a program embedded in a specific apparatus.

The skin characteristic data visualization device 10 may be a mobile terminal. For example, the skin characteristic data visualization device 10 may be implemented in a mobile phone such as a smart phone, a computer, a tablet, a laptop, and the like, but it is merely illustrative, and thus the embodiment is not limited thereto.

Meanwhile, the skin characteristic data visualization device 10 may receive the skin characteristics measured by the measuring device 30 as described later or may directly measure the skin characteristics. For example, the skin characteristic data visualization device 10 may measure skin characteristics through a camera 15 to be described later.

The measuring device 30 may be a skin measuring device that measures skin characteristics. For example, the measuring device 30 may be a DSLR camera, a skin diagnosis device, etc., but it is merely illustrative, and thus the embodiment is not limited thereto.

The measuring device 30 may be connected to the skin characteristic data visualization device 10 by wire or wireless manner. For example, the measuring device 30 may be connected to the skin characteristic data visualization device 10 through Bluetooth or Wi-Fi to communicate wirelessly. The server 50 may store data for converting skin characteristic data into an output form.

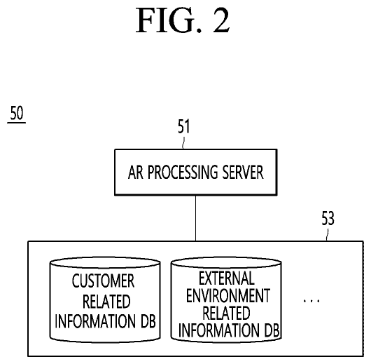

FIG. 2 is a block diagram illustrating a detailed configuration of the server of FIG. 1.

The server 50 may be connected to the skin characteristic data visualization device 10 through the Internet.

The server 50 may include an AR processing server 51 and a DB server 53 providing various databases (DBs).

The AR processing server 51 may perform image processing in which specific data is implemented by the augmented reality (AR).

The DB server 53 may include a customer-related information DB, an external environment-related information DB, and the like.

The customer-related information DB may include information such as customer stress, eating habits, skin care, and the like.

The customer-related information DB may be divided into a customer personal information database, a customer bio database, a VR skin point database, a color, line, and dot setting database for each skin characteristic, a video simulation timeline database, and the like.

The customer personal information database may individually store information on each user.

The customer bio database may store bio information such as skin characteristics for each user.

The VR skin point database may store data for implementing a VR skin according to the user's skin characteristics.

The color, line, and dot setting database for each skin characteristic may store data for setting a color, a line, or a dot applied to a user's facial image according to skin characteristics and measurement information.

The video simulation timeline database may store data for implementing a simulation image implemented by the AR into a video.

Data stored in the color, line, and dot setting database for each skin characteristic and the video simulation timeline database may be data in which data input to at least one Excel file is parsed by an Excel file parsing processor.

The external environment information DB may include information on an external environment such as weather, temperature, humidity, ultraviolet index, and fine dust index, etc.

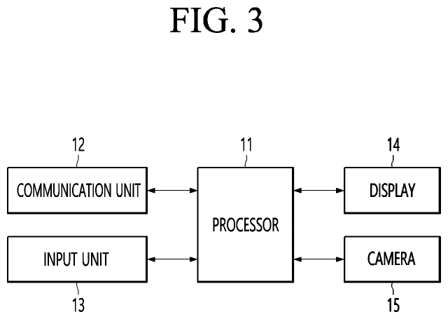

FIG. 3 is a control block diagram of a skin characteristic data visualization device according to an embodiment of the present disclosure.

A skin characteristic data visualization device 10 may include at least some or all of a processor 11, a communication unit 12, an input unit 13, a display 14, and a camera 15.

The processor 11 may perform calculations required to visualize skin characteristic data. The processor 11 may transmit and receive information to and from the communication unit 12, the input unit 13, the display 14, and the camera 15.

The communication unit 12 may transmit and receive information to and from at least some or all of the measuring device 30 or the server 50.

The input unit 13 may receive a user input. The input unit 13 may receive a touch input through the display 14 or may be implemented as a separate physical key button.

The display 14 may visually output skin characteristic data.

The camera 15 may take an image, in particular, a user's skin image.

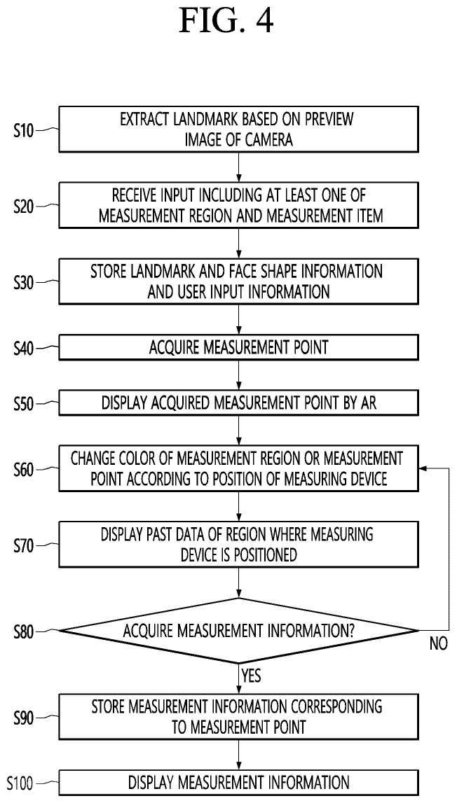

FIG. 4 is a flowchart illustrating a method of operating the skin characteristic data visualization device according to the embodiment of the present disclosure.

The processor 11 may display a measurement guide before starting measurement. That is, when the measurement is started, the processor 11 may first display the measurement guide.

The measurement guide may include measurement conditions. For example, the measurement guide may include measurement conditions such as a skin condition before measurement (e.g., whether a face is bare), a measurement time zone (e.g., a time zone similar to a past measurement time zone, a past measurement time±30 minutes).

The processor 11 may also display a past measurement time when displaying a measurement condition guiding the measurement time zone.

The processor 11 may extract a landmark based on a preview image of the camera 15 (S10).

The processor 11 may control the display 14 to output information guiding the user to capture a skin image through the camera 15. The processor 11 may receive an image photographed through the camera 15.

The processor 11 may acquire the preview image of the camera 15. The preview image may be an image that previews an image to be photographed by the camera 15 before a photographing button is input.

When the processor 11 receives the preview image of the camera 15, the processor 11 may extract the landmark based on the preview image. In particular, when the processor 11 transmits the preview image to the server 50, the server 50 may acquire the landmark by utilizing artificial intelligence (AI) and may transmit information on the acquired landmark to the skin characteristic data visualization device 10. When acquiring the landmark, google ml kit or vision api may be used.

The landmark may refer to at least one region used for face analysis. For example, the landmark may include an eyebrow region, an eye region, a nose region, a mouth region, a philtrum region, a forehead region, and the like.

If the landmark is used when acquiring a measurement point described later, there is an advantage that the measurement point may be more accurately expressed by the augmented reality even though a person has a different face size, shape, and the like.

The processor 11 may receive an input including at least one of a measurement region and a measurement item (S20).

The processor 11 may receive at least one of a measurement region and a measurement item that the user wants to measure skin through the input unit 13.

The measurement region may refer to a position or site measured through the measuring device 30. For example, the measurement region includes eye rims, cheeks, mouth, a T zone, a smile zone, and the like, but it is merely illustrative, and thus the embodiment is not limited thereto.

The measurement item may refer to a measurement target through the measuring device 30. For example, the measurement item includes moisture, elasticity, and the like, but it is merely illustrative, and thus the embodiment is not limited thereto.

The processor 11 may store landmark and face shape information and user input information (S30).

The processor 11 may store landmark and face shape information acquired through the preview image of the camera 15 and user input information received in step S20. Although not shown in FIG. 3, the skin characteristic data visualization device 10 may also include a separate memory (not shown) for storing the landmark and face shape information and the user input information.

The processor 11 may acquire a measurement point (S40).

The measurement point may refer to a point to be measured by the measuring device 30.

The processor 11 may acquire the measurement point from an internal program or the server 50.

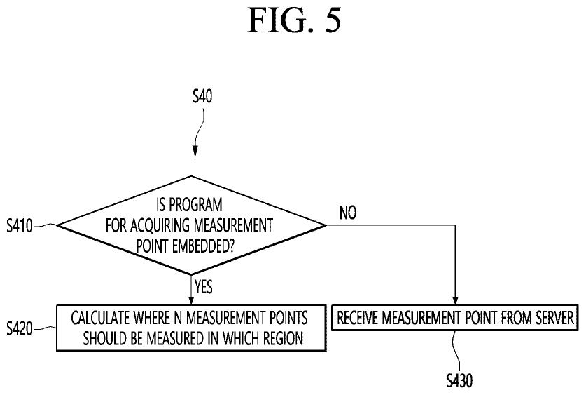

FIG. 5 is a flowchart illustrating a method in which the skin characteristic data visualization device acquires a measurement point. In particular, FIG. 5 is a flowchart in which step S40 of FIG. 4 is embodied.

The processor 11 may determine whether a program for acquiring the measurement point is embedded (S410). That is, the processor 11 may determine whether the program for acquiring the measurement point is stored therein.

When the program is embedded therein, the processor 11 may calculate where N measurement points should be measured in which region (S420).

When the program is not embedded, the processor 11 may receive measurement points from the server 50 (S430).

That is, when the program is not embedded, the server 50 may calculate where N measurement points should be measured in which region, and the processor 11 may receive the measurement points from the server 50.

The processor 11 may calculate a number of measurement points according to user characteristics acquired from the database 20.

The processor 11 may display the acquired measurement points by the augmented reality (AR) (S50).

Specifically, the processor 11 may display to overlap the measurement points on the preview image of the camera 15 on the display 14. That is, the processor 11 may display an image in which a measurement point appears in a user's facial image through an AR technology.

In addition, when displaying the measurement points, the processor 11 may further display a position of the measurement device 30 or the measurement region by the AR. The position of the measuring device 30 may be a tip position of the end of the measuring device 30.

That is, the processor 11 may display the measurement position of the measuring device 30 through a camera preview using the AR technology.

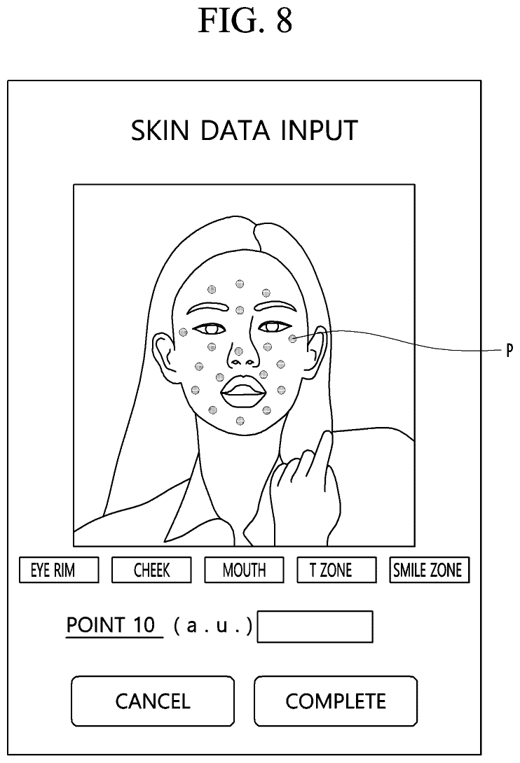

Since such an image is shown in an example of FIG. 8, it will be described in detail in FIG. 8.

The processor 11 may change a color of the measurement region or measurement point according to the position of the measuring device 30 (S60).

The processor 11 may recognize the position of the measuring device 30 appearing on the preview image of the camera 15. Based on the position of the measuring device 30, when the measuring device 30 is positioned in the measuring region or the measuring point, the processor 11 may change a color of the measuring region or measuring point.

The processor 11 may display past data of a region where the measuring device 30 is positioned (S70).

When there is a past measurement record at the measurement point where the measuring device 30 is positioned, the processor 11 may control the display 14 so as to display corresponding past data.

The past data may include a simulation image implemented by the AR by overlapping a VR skin visualizing skin characteristics according to past measurement information on a user's real facial image.

The processor 11 may determine whether measurement information has been acquired (S80).

The measurement information may refer to a measurement value of the measuring device 30. The processor 11 may receive the measurement value of the measuring device 30 from the measuring device 30 or may receive input for the measurement value through the input unit 13.

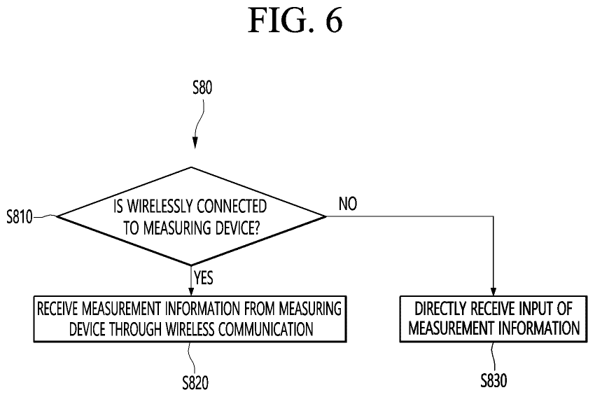

FIG. 6 is a flowchart illustrating a method of acquiring measurement information by the skin characteristic data visualization device according to the embodiment of the present disclosure. FIG. 6 may be a flowchart in which step S80 of FIG. 4 is embodied.

The processor 11 may determine whether the skin characteristic data visualization device is wirelessly connected to the measuring device 30 (S810).

For example, the processor 11 may determine whether the skin characteristic data visualization device is connected to the measuring device 30 through Bluetooth or the skin characteristic data visualization device is connected to the measuring device 30 through the same Wi-Fi.

When the skin characteristic data visualization device is wirelessly connected to the measuring device 30, the processor 11 may receive measurement information from the measuring device 30 through wireless communication (S820).

When the skin characteristic data visualization device is not wirelessly connected to the measuring device 30, the processor 11 may directly receive an input of measurement information (S830).

That is, when the skin characteristic data visualization device is not wirelessly connected to the measuring device 30, the processor 11 may acquire a measurement value directly input by the user through the input unit 13. When the skin characteristic data visualization device 10 is not wirelessly connected to the measuring device 30, the user may directly input the measured value of the measuring device 30 to the input unit 13.

FIG. 4 will be described again.

When acquiring measurement information, the processor 11 may store measurement information corresponding to a measurement point (S90).

The processor 11 may correspond and store the measurement information to each measurement point.

The processor 11 may display the measurement information (S100).

The processor 11 may control the display 14 so as to display the measurement information.

The processor 11 may display the measurement information as a simulation image. The simulation image may be an image acquired by visualizing skin characteristics on a user's real facial image using the AR technology. Visualizing the skin characteristics may refer to generating a VR skin that virtually implements the user's skin according to the measurement information.

The processor 11 may display past measurement information as well as current measurement information as the simulation image.

In addition, the processor 11 may display the measurement information from the past to the present or from the past to a specific time point as the simulation image in a moving image corresponding to the passage of time. In this case, the processor 11 may display a change in skin characteristics as the simulation image according to a measurement date and a measurement value in the past.

The processor 11 may generate the VR skin according to the skin characteristics and display it on the user's real facial image.

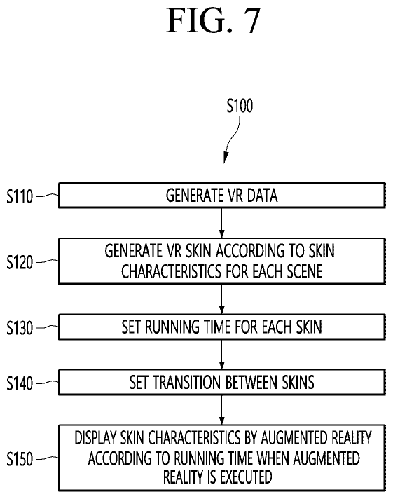

FIG. 7 is a flowchart illustrating a method of displaying measurement information by the skin characteristic data visualization device according to the embodiment of the present disclosure. That is, FIG. 7 is a flowchart in which step S100 of FIG. 4 is embodied.

The processor 11 may generate VR data (S110).

The VR data may refer to data for generating a virtual skin representing skin characteristics of a user according to the measurement information, that is, the VR skin.

The processor 11 may generate the VR skin according to the skin characteristics for each SCENE (S120).

The processor 11 may generate the VR skin in which the skin characteristics of the user according to the measurement information of the measuring device 30 is displayed and may generate the VR skin for each SCENE. Here, the VR skin for each SCENE may refer to a VR skin for each measurement region, each measurement item, or each period.

The processor 11 may set a running time for each skin (S130).

The processor 11 may set a running time for each generated VR skin, and the running time may refer to a displayed playback time.

The processor 11 may set TRANSITION between skins (S140).

TRANSITION between skins may refer to an effect displayed between the VR skins. TRANSITION may refer to a transition effect between scenes.

That is, in step S140, the processor 11 may set the transition effect between the scenes to be applied when displaying the simulation image.

The processor 11 may display the skin characteristics by augmented reality according to the running time when the augmented reality is executed (S150).

That is, the processor 11 may implement the simulation image by the AR by overlapping the generated VR skin on the user's real facial image. Specifically, the processor 11 may express a first VR skin according to measurement information of a first time point on the user's real facial image and then may express a second VR skin according to measurement information of a second time point on the user's real facial image. For example, the processor 11 may overlap and display the first VR skin with acne at a 19^{th} measurement point on the user's facial image, and then the processor 11 may overlap and display a second VR skin without at the 19^{th} measurement point on the user's facial image.

Next, various screens related to an operation of a skin characteristic data visualization device 10 according to an embodiment of the present disclosure will be described with reference to FIGS. 8 to 22.

FIG. 8 is an example of a screen displaying measurement points by the AR by the skin characteristic data visualization device according to the embodiment of the present disclosure.

When the processor 11 displays the measurement points by the AR, as shown in FIG. 8, a display 14 may display an image in which at least one measurement point P overlaps a user's facial image.

When a face moves, the processor 11 may track the movement of the face and may move the measurement point P together.

When displaying the measurement point P, the display 14 may display a measurement region together. As shown in the example of FIG. 8, the display 14 may display icons representing measurement regions such as eye rims, cheeks, mouth, T zone, and smile zone.

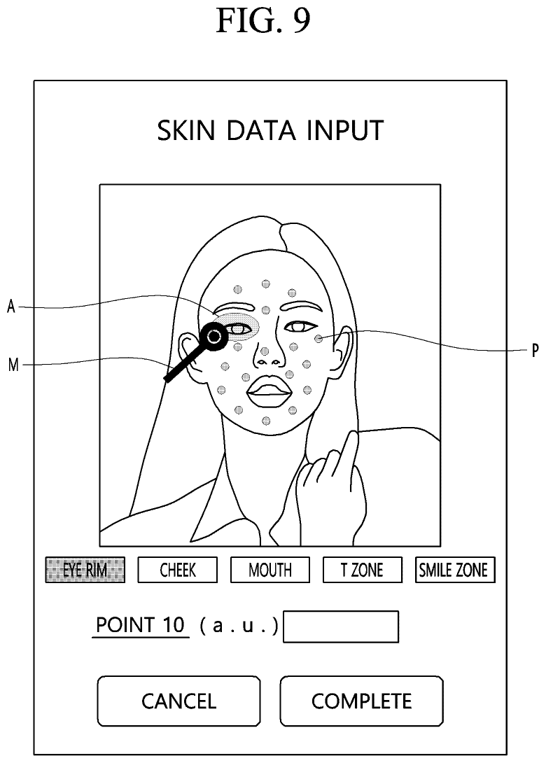

FIG. 9 is an example of a screen in which the skin characteristic data visualization device according to the embodiment of the present disclosure displays a measurement point, a measurement region, and a position of a measurement device by the AR.

When an icon representing the measurement region is selected, the processor 11 may control the display 14 so as to display a measurement region indicator A indicating the selected measurement region. In this case, the processor 11 may calculate a size of the measurement region as a ratio (percentage) in proportion to a face size. For example, the processor 11 may calculate a size of the eye rim region as 10 cm when a length between the left and right cheeks is 20 cm and may calculate the size of the eye rim region as 15 cm when the length between the left and right cheeks is 30 cm.

In addition, the processor 11 may change a color of a measurement point to be measured currently among several measurement points.

In addition, the processor 11 may control the display 14 so as to further display a measuring device indicator M indicating the position of the measuring device 30 together with the measuring region indicator A.

Meanwhile, the processor 11 may deactivate a point input window when the measuring device 30 is not positioned at the measuring point P. In addition, the processor 11 may activate the point input window when the measuring device 30 is positioned at the measuring point P. The processor 11 may determine whether the measuring device 30 is positioned at the measuring point P based on an area in which the position of the measuring device indicator M and the measuring point P intersect.

The processor 11 may wirelessly receive a measurement value input to the point input window from the measuring device 30 or directly receive input for the measurement value through the input unit 13.

Meanwhile, the processor 11 may acquire the measurement value at another point not guided to the measurement point P, and in this case, a new measurement point P may be created.

The processor 11 may control the display 14 so as to further display past data when there is a past measurement record corresponding to the measurement point P at which the measuring device 30 is positioned. The past data may include a measurement value and a simulation according to the measurement value.

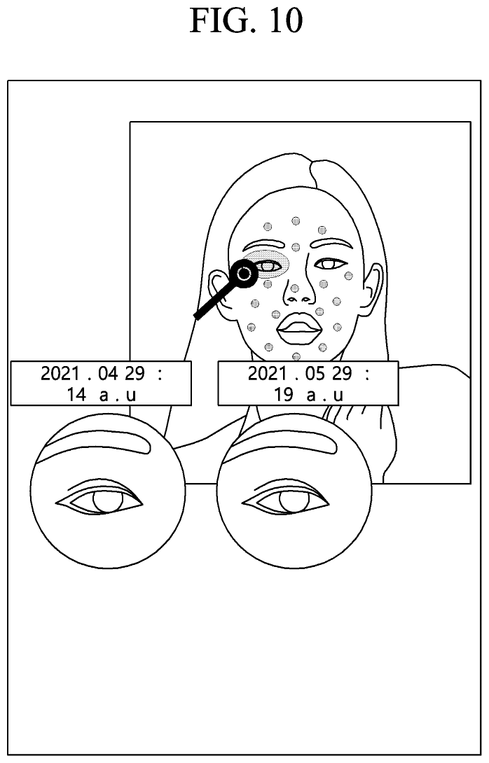

FIG. 10 is an exemplary view illustrating a screen on which the skin characteristic data visualization device according to the embodiment of the present disclosure displays past data.

The processor 11 may indicate on the display 14 at least one piece of past data corresponding to the measurement point P where the measuring device 30 is positioned. As shown in the example of FIG. 10, the processor 11 may display a measurement date, a measurement value, and a simulation according to the measurement value on the display 14 together.

The processor 11 may enlarge the measurement region including the corresponding measurement point at a predetermined ratio to display the simulation according to the measurement value.

Meanwhile, the processor 11 may differently calculate a number of measurement points P required to be measured in the measurement region according to a face size.

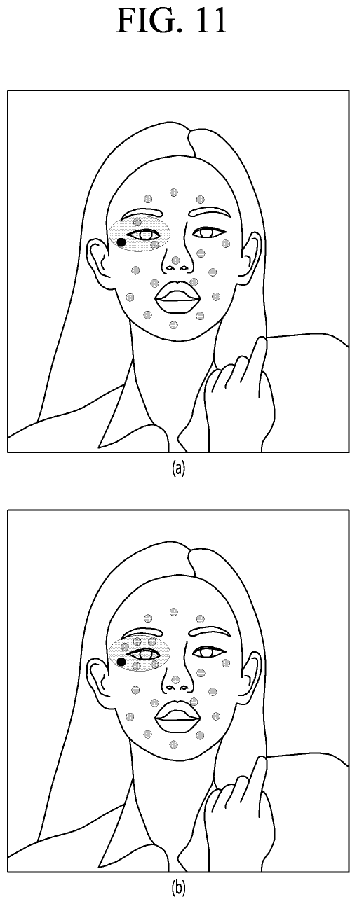

FIG. 11 is a view illustrating a state in which the skin characteristic data visualization device according to the embodiment of the present disclosure varies the number of measurement points according to the face size.

For example, when the face size is smaller than a reference size, the processor 11 may calculate the number of measurement points of the measurement region corresponding to the eye rim as three, and when the face size is greater than the reference size, the processor 11 may calculate the number of measurement points of the measurement region corresponding to the eye rim as six. In addition, the processor 11 may display the measuring point P differently according thereto, FIG. 11A is an image illustrating the measuring point P when the face size is smaller than the reference size, and FIG. 11B is an image illustrating the measurement point P when the face size is greater than the reference size.

Meanwhile, when only measurement information on a part of a plurality of measurement points P is acquired, the processor 11 may predict a measurement value corresponding to the measurement point P in which the measurement information does not exist through interpolation or machine learning.

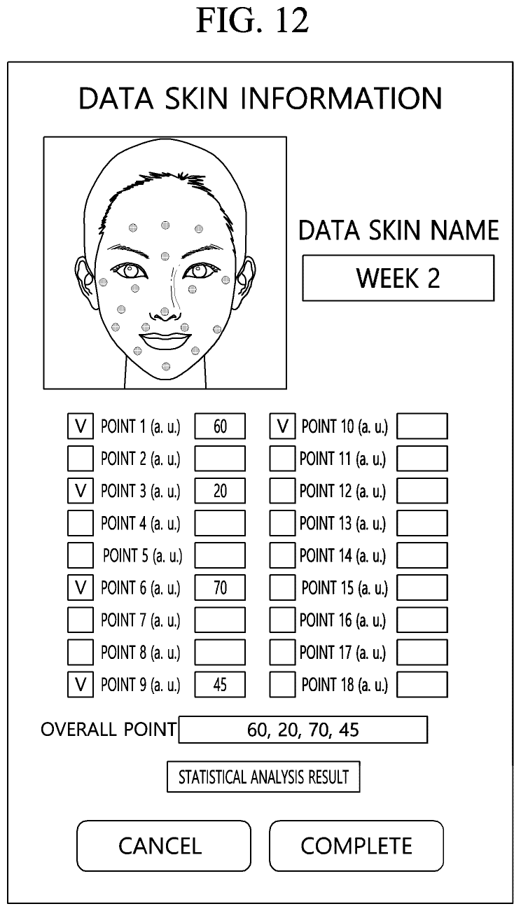

FIG. 12 is an example of a screen on which measurement information is displayed in the skin characteristic data visualization device according to the embodiment of the present disclosure.

For example, referring to the example of FIG. 12, the processor 11 may predict a value acquired by adding a value acquired by multiplying a measurement value of a third measurement point by 0.2 and a value acquired by multiplying a measurement value of a sixth measurement point by 0.3 and then dividing the value by 2 as a measurement value of a fifth measurement point, where 0.2 may be a calculated value according to a distance between the third measurement point and the fifth measurement point, and 0.3 may be a calculated value according to a distance between the sixth measurement point and the fifth measurement point.

As another example, based on a result of learning data of a woman in her twenties, when the measurement value corresponding to the third measurement point of the woman in her twenties is 20 and the measurement value corresponding to the sixth measurement point is 70, the processor 11 may calculate the measurement value corresponding to the fifth measurement point as 40.

Next, Table 1 below is an example of data used when the processor 11 generates a simulation. In particular, Table 1 is an example of data used when the processor 11 creates a VR skin. For example, when selecting elasticity, selecting network tension simulation, and the ninth measurement point is 8, it corresponds to the index 80, and the processor 11 may create a VR skin having sagging of 30, and tension of 70 around the ninth measurement point.

TABLE 1

| | Network | | | | |
|---|---|---|---|---|---|
| | Tension | Thickness | Shining | Color | Color mapping |
| elasticity: 1 | sagging 100:tension 0 | 1 pt | brightness 1 | BLUE100:RED0 | BLUE100:RED0 |
| moisture: 10 | | | | | |
| shine: 10 | | | | | |
| soothing: 10 | | | | | |
| index: 10 | | | | | |
| elasticity: 2 | sagging 90:tension 10 | 2 pt | brightness 2 | BLUE90:RED10 | BLUE90:RED10 |
| moisture: 20 | | | | | |
| shine: 20 | | | | | |
| soothing: 20 | | | | | |
| index: 20 | | | | | |
| elasticity: 3 | sagging 80:tension 20 | 3 pt | brightness 3 | BLUE80:RED20 | BLUE80:RED20 |
| moisture: 30 | | | | | |
| shine: 30 | | | | | |
| soothing: 30 | | | | | |
| index: 30 | | | | | |
| elasticity: 4 | sagging 70:tension 30 | 4 pt | brightness 4 | BLUE70:RED30 | BLUE70:RED30 |
| moisture: 40 | | | | | |
| shine: 40 | | | | | |
| soothing: 40 | | | | | |
| index: 40 | | | | | |
| elasticity: 5 | sagging 60:tension 40 | 5 pt | brightness 5 | BLUE60:RED40 | BLUE60:RED40 |
| moisture: 50 | | | | | |
| shine: 50 | | | | | |
| soothing: 50 | | | | | |
| index: 50 | | | | | |
| elasticity: 6 | sagging 50:tension 50 | 6 pt | brightness 6 | BLUE50:RED50 | BLUE50:RED50 |
| moisture: 60 | | | | | |
| shine: 60 | | | | | |
| soothing: 60 | | | | | |
| index: 60 | | | | | |
| elasticity: 7 | sagging 40:tension 60 | 7 pt | brightness 7 | BLUE40:RED60 | BLUE40:RED60 |
| moisture: 70 | | | | | |
| shine: 70 | | | | | |
| soothing: 70 | | | | | |
| index: 70 | | | | | |
| elasticity: 8 | sagging 30:tension 70 | 8 pt | brightness 8 | BLUE30:RED70 | BLUE30:RED70 |
| moisture: 80 | | | | | |
| shine: 80 | | | | | |
| soothing: 80 | | | | | |
| index: 80 | | | | | |
| elasticity: 9 | sagging 30:tension 80 | 9 pt | brightness 9 | BLUE20:RED80 | BLUE20:RED80 |
| moisture: 90 | | | | | |
| shine: 90 | | | | | |
| soothing: 90 | | | | | |
| index: 90 | | | | | |
| elasticity: 10 | sagging 10:tension 90 | 10 pt | brightness 10 | BLUE10:RED90 | BLUE10:RED90 |
| moisture: 100 | | | | | |
| shine: 100 | | | | | |
| soothing: 100 | | | | | |
| index: 100 | | | | | |

The processor 11 may generate an AR skin using measurement information for each time period (e.g., week 0, week 1, and week 3).

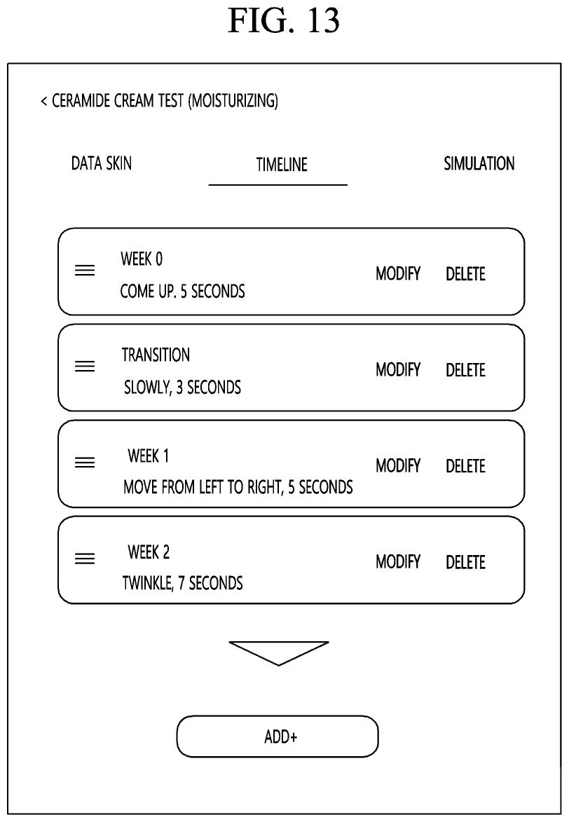

FIG. 13 is a diagram illustrating a state in which the skin characteristic data visualization device according to the embodiment of the present disclosure displays measurement information for each period in a timeline.

As shown in an example of FIG. 13, the processor 11 may display the measurement information for each period in a timeline in chronological order and may set a TRANSITION between each period.

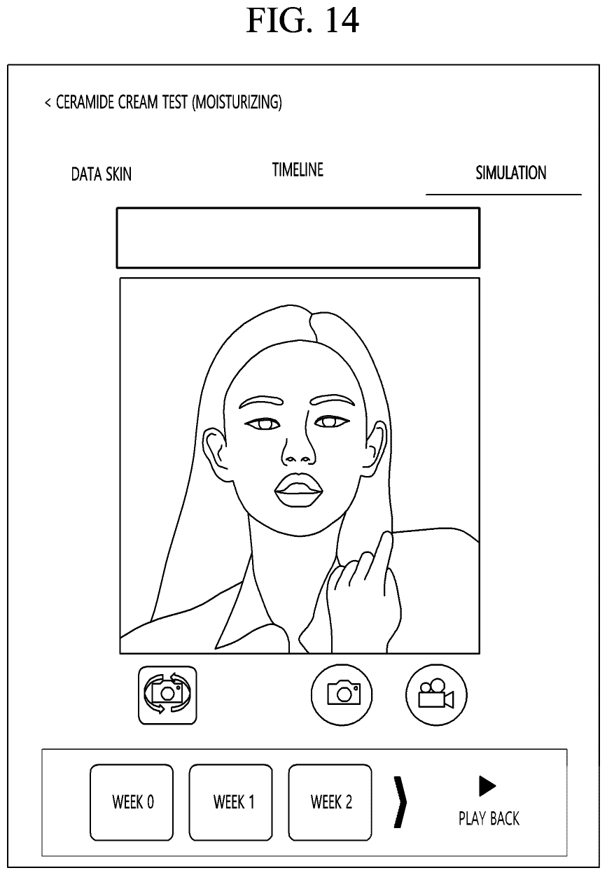

FIG. 14 is a diagram illustrating a state in which the skin characteristic data visualization device according to the embodiment of the present disclosure displays a simulation.

As in an example of FIG. 14, the processor 11 may display an icon representing each period together with a simulation image.

For example, the processor 11 may display by simulating a face that was dry at week 0 for 7 seconds, a face that was somewhat improved at week 1 for 7 seconds, and a face that was moist at week 2 for 7 seconds.

In addition, the processor 11 may provide external environment information that affects a skin condition. For example, the processor 11 may display external environment information that affects the skin condition together with the simulation.

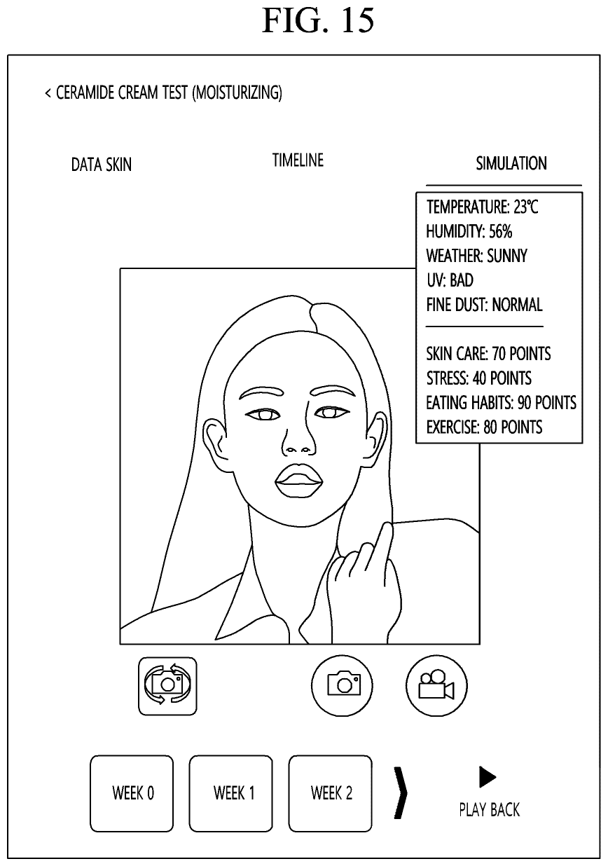

FIG. 15 is a diagram illustrating a state in which the skin characteristic data visualization device according to the embodiment of the present disclosure displays the simulation together with monitor information.

The processor 11 may display monitor information together with the simulation. The monitor information may include external environment information such as weather, temperature, humidity, UV index, and fine dust index, which may be acquired from an external environment related information DB.

Information such as temperature, humidity, skin care, etc. may be displayed as numbers or degrees (e.g., bad, normal, good), and the numbers or degrees may be displayed in a form of a graph (e.g., bar graph).

The graph may further include monitor information about a time point corresponding to the simulation and a past time point or a future time point. In this case, an indicator such as a cursor indicating a time point corresponding to the simulation may be further included.

The external environment-related information DB may provide an average temperature, an average humidity, and an average weather during the skin measurement period to the skin characteristic data visualization device 10.

In addition, the processor 11 may receive ultraviolet light information from the external environment related information DB. For example, when the skin characteristic data visualization device 10 is a mobile phone, a user's movement line is grasped by a GPS of a mobile phone to acquire a date and time that the user traveled outside, thereby acquiring ultraviolet light information at the corresponding place, date and time.

In addition, the monitor information may include customer information acquired through questionnaires, etc., which may include stress, eating habits, skin care information, exercise information, sleep time, schedule information, etc. of the customer, which may be acquired from the customer related information DB.

For example, the skin care information may include information on cosmetics being used by the user.

For example, when the skin characteristic data visualization device 10 is the mobile phone, the processor 11 may calculate the sleep time by using an acceleration sensor and usage time of the mobile phone.

In addition, when the skin characteristic data visualization device 10 is the mobile phone, the processor 11 may acquire the user's schedule information (e.g., whether the user went out or not, a user's out date, etc.) through a schedule-related application.

Therefore, the processor 11 may acquire whether the user is exposed to ultraviolet rays, sleep time, and the like for each date based on the user's schedule information to display an improvement effect according to the cosmetic information in use by simulation. That is, when specific cosmetics is continuously being used, the processor 11 may display by simulation so as to visually guide improvements according to corresponding cosmetics.

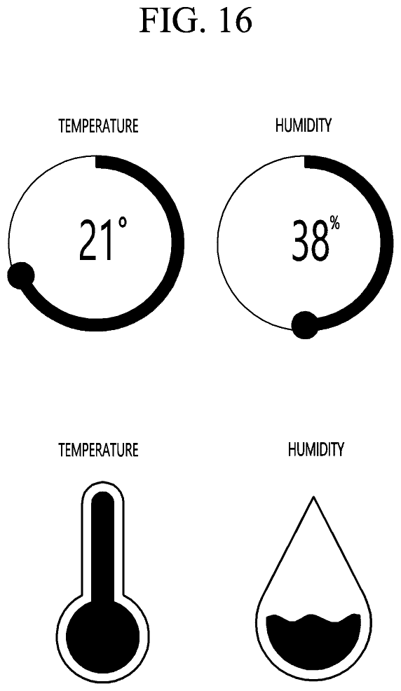

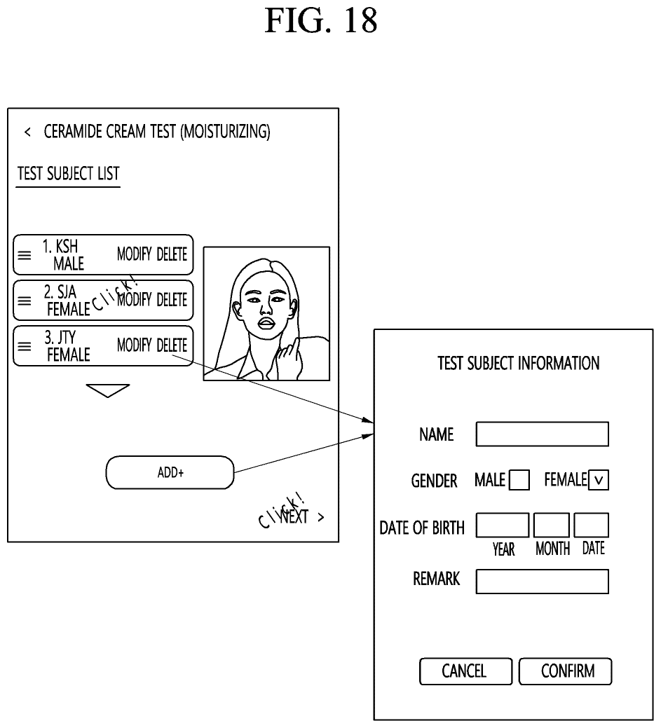

In FIGS. 18 to 22, a method for displaying the improvements in moisturizing through ceramide cream has been described as an example, but the embodiment is not limited thereto, and the processor 11 may provide improvements for various cosmetics and various effects (e.g., wrinkles, soothing, whitening, etc.) through the method shown in FIGS. 18 to 22. Meanwhile, the monitor information as described in FIG. 16 may be displayed as a monitor information icon as shown in FIG. 16.

FIG. 16 is a diagram illustrating a state in which the skin characteristic data visualization device according to the embodiment of the present disclosure displays a monitor information icon.

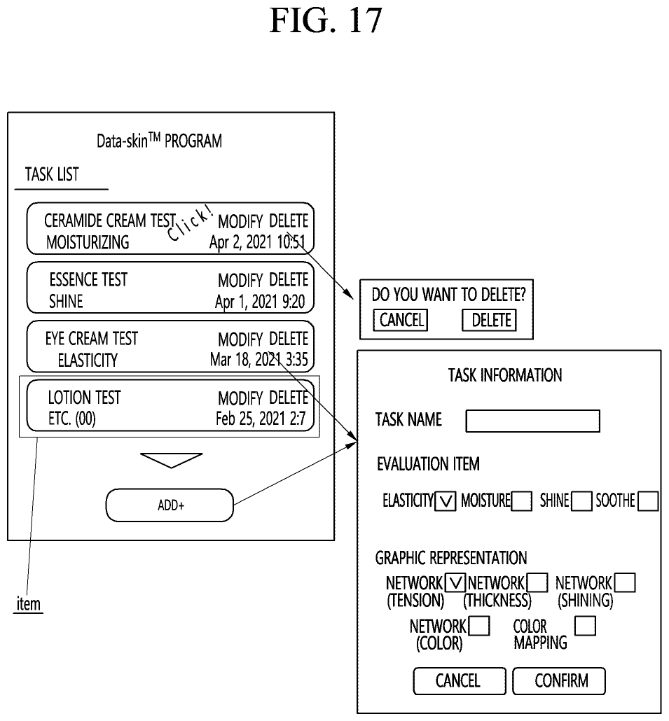

FIG. 17 is a diagram illustrating a method of receiving task information by the skin characteristic data visualization device according to the embodiment of the present disclosure.

The task may refer to measuring an effect over time for each cosmetic.

The processor 11 may display a task list composed of at least one task item. Each of the task items included in the task list may include a task name, skin characteristics, date and time of creation. The processor 11 may dispose the most recently progressed task upward by sorting the tasks in the latest order. When ‘delete’ on the right side of each task item is selected, the processor 11 may generate a pop-up and delete corresponding task subject data. When ‘Modify’ on the right side or ‘+ Add’ at the bottom of each task item is selected, the processor 11 may generate a task information input pop-up. When each task item is selected, the processor 11 displays a list of test subjects for the corresponding task, which may be the same as an example of FIG. 18.

FIG. 18 is a diagram illustrating a method of receiving test subject information by the skin characteristic data visualization device according to the embodiment of the present disclosure.

The processor 11 may display a list of test subjects and display names (or initials), gender, and age on individual task items. When ‘delete’ on the right side of each task item is selected, the processor 11 may generate a pop-up and delete corresponding test subject data. The processor 11 may generate a test subject information input pop-up by pressing the ‘Modify’ button on the right side or the ‘+ Add’ button at the bottom of each task item.

When the test subject item is selected, the processor 11 may display a stored photograph on the right screen and may activate a ‘Next’ button at the bottom.

The processor 11 may change an order by dragging and dropping the left side of each task item.

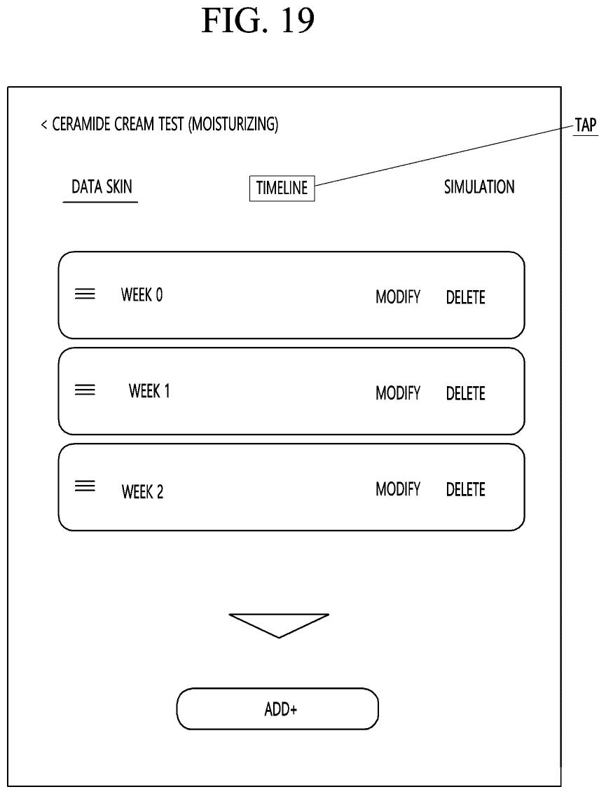

FIG. 19 is a diagram illustrating a method of displaying a data skin list by the skin characteristic data visualization device according to the embodiment of the present disclosure.

The data skin may refer to a data-based effect and a time point to be overlaid during an augmented reality (AR) effect.

The processor 11 may first activate ‘data skin’ among three top tabs and display a data skin list. The data skin list may include at least one data skin item. The processor 11 may display a name for each data skin item, and when ‘delete’ is selected on the right side, the processor 11 may generate the pop-up and delete the corresponding data skin. The processor 11 may generate a data skin information input pop-up when a ‘modify’ button on the right side of each data skin item or a ‘+ add’ button at the bottom is selected. The processor 11 may change the order by dragging and dropping the left side of each data skin item.

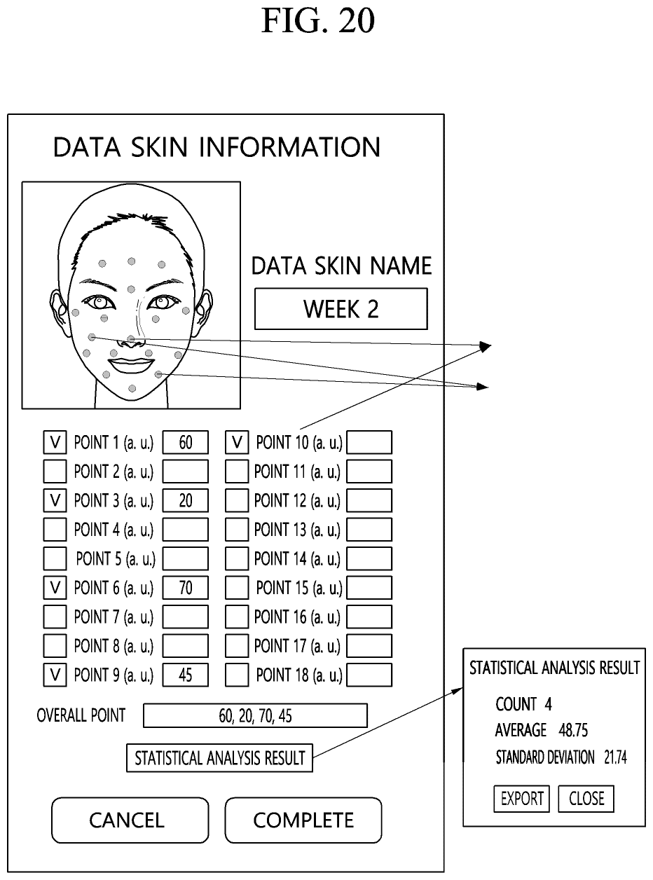

FIG. 20 is an exemplary view of the data skin information input pop-up displayed by the skin characteristic data visualization device according to the embodiment of the present disclosure.

The processor 11 may display a plurality of measurement points together with an illustration (or a front cut of a model). The number of measurement points may be 18, but it is merely illustrative and may be excluded when a numerical value is not input.

When an icon (‘point n’) representing a nth measurement point is selected, the processor 11 may activate a corresponding measurement point while changing a border color of the corresponding measurement point on an image.

The processor 11 may distinguish a color between a measurement point where a measurement value is input and a measurement point where a measurement value is not input.

The processor 11 may display an input window capable of inputting measurement values of all measurement points at once at the bottom of the measurement value input field for each measurement point. In this case, the processor 11 may divide the data with commas or spaces, and when the input is completed, a numerical value may be substituted into the activated corresponding measurement point.

Alternatively, the processor 11 may immediately apply the numerical value when external input data (such as an Excel file) is loaded.

When an input field of the data skin name is displayed at the top right side and the ‘Statistical Analysis Result’ button at the bottom is selected, the processor 11 may generate a pop-up with information such as a number of data, an average, and a standard deviation, and a result displayed in the pop-up allows so as to export the entire data in a file form such as an abbreviated form, .xlsx, or the like.

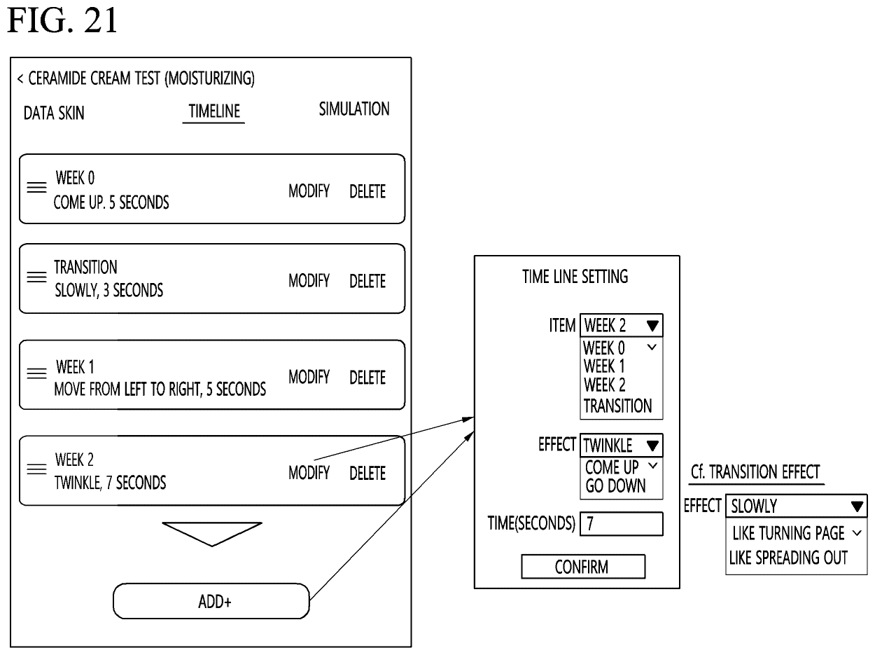

FIG. 21 is an exemplary diagram illustrating a method of setting a timeline by the skin characteristic data visualization device according to the embodiment of the present disclosure.

The processor 11 may display the entire timeline and may display a data skin name, an effect, and a time in an individual item. When ‘delete’ on the right side of each item is selected, the processor 11 may display a pop-up to delete the corresponding task. The processor 11 may display a timeline setting input pop-up when the ‘Modify’ button on the right side of each item or the ‘+ Add’ button at the bottom of the list is selected. The processor 11 may change the order by dragging and dropping the left side of each item.

The processor 11 may select stored a data skin or TRANSITION, and TRANSITION may be an effect between data skins. When the data skin is selected, the processor 11 may set an effect and a time (in seconds) for AR to be implemented in an image. Even when TRANSITION is selected, the processor 11 may set an effect and a time, and in this case, it is possible to select effects different from the data skin.

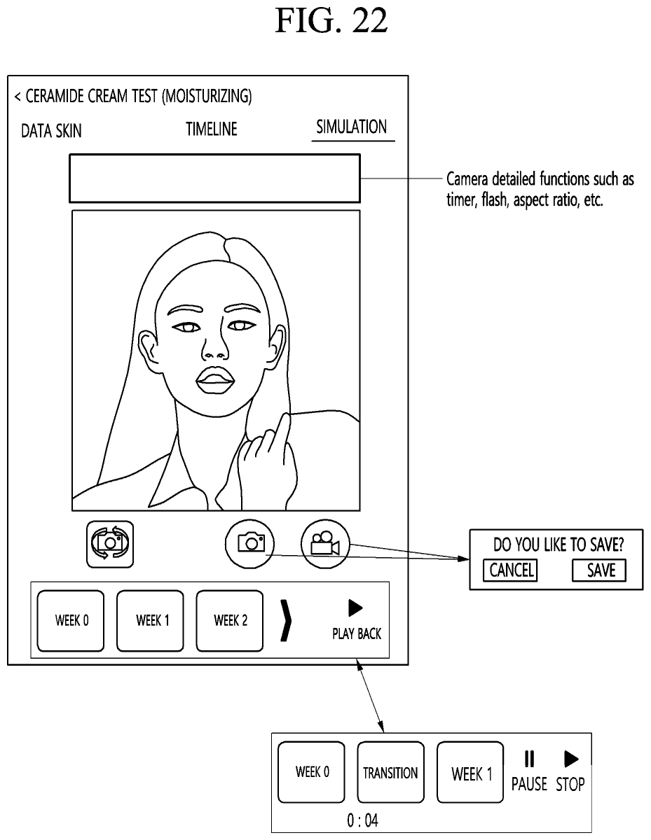

FIG. 22 is an exemplary diagram illustrating a method of setting a simulation by the skin characteristic data visualization device according to the embodiment of the present disclosure.

The processor 11 may overlap and display AR representing skin characteristics according to a measured value on a face on a screen in real-time. The processor 11 may apply immediately when an individual data skin item is selected at the bottom and may apply the effect for a previously set time when a ‘play’ button at the bottom right is selected. The processor 11 may change the ‘play’ button to ‘pause’ and ‘stop’ buttons during playback. The processor 11 may perform capture when a camera icon is selected and may perform image recording when a video icon is selected.

The present disclosure described above may be implemented as computer-readable codes in a medium on which a program is recorded. The computer-readable medium includes all kinds of recording devices in which data readable by a computer system is stored. Examples of the computer-readable medium include a hard disk drive (HDD), a solid state disk (SSD), a silicon disk drive (SDD), a ROM, a RAM, a CD-ROM, a magnetic tape, a floppy disk, and an optical data storage, etc. In addition, the computer may include a controller of a cosmetic manufacturing device or a controller of a terminal. Therefore, the above detailed description should not be construed in a limiting sense in all respects, and should be considered as examples. The scope of the present invention should be determined by rational interpretation of the appended claims, and encompasses all alterations falling within the equivalent scope of the appended claims.

The above-described descriptions are merely an example of the technological spirit of the present invention, and various modifications and variations may be made by those having ordinary skill in the art to which the present invention pertains without departing from the essential characteristics of the present invention.

Therefore, the embodiments disclosed in the present invention are not intended to limit the technological spirit of the present invention, but the embodiments are intended to describe, and the spirit and scope of the present invention is not limited by such embodiments.

The protection scope of the present invention should be construed by the following claims, and all technological spirits within the equivalent scope thereof should be construed as being included in the scope of right of the present invention.

What is claimed is:

1. A skin characteristic data visualization device comprising:
a camera;
a display displaying a preview image of the camera; and
a processor that acquires a measurement point which is a position of a skin to be measured by a measuring device,
wherein the processor displays the measurement point on the preview image by an augmented reality (AR).

2. The skin characteristic data visualization device of claim 1, wherein the processor changes a color of the measurement point when the measuring device is positioned at the measurement point.

3. The skin characteristic data visualization device of claim 1, wherein the processor further displays a position of the measuring device on the preview image by the AR.

4. The skin characteristic data visualization device of claim 1, wherein the processor activates a window for inputting a measurement value when the measuring device is positioned at the measurement point and deactivate a window for inputting the measurement value when the measuring device is not positioned at the measurement point.

5. The skin characteristic data visualization device of claim 1, wherein the processor further displays past data when there is a past measurement record at the measurement point where the measuring device is positioned.

6. The skin characteristic data visualization device of claim 1, wherein the processor displays a number of measurement points differently according to a size of a user's face on the preview image.

7. The skin characteristic data visualization device of claim 1, wherein the processor further displays a simulation image representing a change in skin characteristics according to a past measurement date and a past measurement value.

8. The skin characteristic data visualization device of claim 7, wherein the processor further displays monitor information together with the simulation image, and
the monitor information includes external environment information and customer information.

9. The skin characteristic data visualization device of claim 7, wherein when displaying the simulation image, the processor set a transition effect between scenes.

10. The skin characteristic data visualization device of claim 1, further comprising a communication unit wirelessly connected to the measuring device, wherein the processor receives measurement information corresponding to the measuring point from the measuring device.

11. A skin characteristic data visualization system comprising:
a measuring device; and
a skin characteristic data visualization device communicating with the measuring device,
wherein the skin characteristic data visualization device includes
a camera,
a display displaying a preview image of the camera, and
a processor acquiring a measurement point which is a position of a skin to be measured by a measuring device,
wherein the processor displays the measurement point on the preview image by an augmented reality (AR).

12. The skin characteristic data visualization system of claim 11, wherein the processor changes a color of the measurement point when the measuring device is positioned at the measurement point.

13. The skin characteristic data visualization system of claim 11, wherein the processor further displays a position of the measuring device on the preview image by the AR.

14. The skin characteristic data visualization system of claim 11, wherein the processor activates a window for inputting a measurement value when the measuring device is positioned at the measurement point and deactivates a window for inputting the measurement value when the measuring device is not positioned at the measurement point.

15. The skin characteristic data visualization system of claim 11, wherein the processor further displays past data when there is a past measurement record at the measurement point where the measuring device is positioned.

16. The skin characteristic data visualization system of claim 11, wherein the processor displays a number of measurement points differently according to a user's face size on the preview image.

17. The skin characteristic data visualization system of claim 11, wherein the processor further displays a simulation image representing a change in skin characteristics according to a past measurement date and a past measurement value.

18. The skin characteristic data visualization system of claim 17, wherein the processor further displays monitor information together with the simulation image, and
the monitor information includes external environment information and customer information.

19. The skin characteristic data visualization system of claim 17, wherein when displaying the simulation image, the processor sets a transition effect between scenes.

20. The skin characteristic data visualization system of claim 11, wherein the skin characteristic data visualization device further includes a communication unit wirelessly connected to the measuring device, and
the processor receives measurement information corresponding to the measuring point from the measuring device.
